(19) United States
(12) Patent Application Publication
(10) Pub. No.: US 2010/0063251 A1
(43) Pub. Date: Mar. 11, 2010

(54) COMPOSITIONS AND METHODS FOR TREATMENT OF CHRONIC FATIGUE SYNDROME AND NEURODEGENERATIVE DISEASES

(76) Inventor: Dorian Bevec, Germering (DE)

Correspondence Address: TUROCY & WATSON, LLP, 127 Public Square, 57th Floor, Key Tower, CLEVELAND, OH, 44114, US

(21) Appl. No.: 12/440,979

(22) PCT Filed: Sep. 14, 2006
(86) PCT No.: PCT/CH2006/000493

§ 371 Date: Nov. 12, 2009

(51) Int. Cl. C07K 7/08

(52) U.S. Cl. 530/327

(57) ABSTRACT

The present invention relates to use of pharmaceutical formulations of a-MSH for the treatment of chronic fatigue syndrome and neurodegenerative diseases.

### FIELD OF INVENTION

The present invention relates to pharmaceutical compositions of α-Melanocyte-stimulating hormone (α-MSH).

### BACKGROUND OF THE INVENTION

The melanocyte-stimulating hormones (collectively referred to as MSH) are a class of peptide hormones produced by cells in the intermediate lobe of the pituitary gland. They stimulate the production and release of melanin (melanogenesis) by melanocytes in skin and hair. MSH is also produced by a subpopulation of neurons in the arcuate nucleus of the hypothalamus. MSH released into the brain by these neurons has effects on appetite and sexual arousal.

Melanocyte-stimulating hormones belong to a group called the melanocortins. Melanocortins are bioactive peptides that are widely expressed in the CNS and in various peripheral tissues. These peptides are involved in the regulation of important physiological functions including food intake, energy homeostasis, and immune function.

The melanocortins comprise a group of natural peptides, all of which are derived from the precursor molecule propiomelanocortin (POMC). POMC is a polyhormone that can give rise to at least 8 distinct peptides whose biologic roles are incompletely delineated Cleavage at tetrabasic sites is an important regulatory step in the processing of POMC in the pituitary, where tissue-specific cleavage at the LysLysArgArg site in POMC produces either adrenocorticotropin (ACTH), alpha-melanocyte-stimulating hormone (α-MSH), beta-MSH and gamma-MSH. Historically, POMC was thought to be produced solely by pituitary cells, but it has become apparent that POMC messenger RNA (mRNA) or POMC-derived peptides are expressed in extrapituitary tissues, such as the arcuate nucleus of the hypothalamus, the commissural nucleus of the brain stem, and the skin. In the spinal cord, immunoreactivity for the POMC-derived peptides ACTH and α-MSH has been detected in the dorsal horn and lamina X. Moreover, POMC-derived peptides were also detected in lymphocytes, monocytes, Langerhans cells, and epithelial cells. α-MSH is well known for its role in the control of melanogenesis in pigmentary cells. However, recent studies demonstrated a potent and broad spectrum of activities as an antipyretic, antimicrobial, anti-inflammatory, immunomodulatory, and a regulator of sexual function peptide.

The different melanocyte-stimulating hormones have the following amino acid sequences:

alpha-MSH:
Ac-Ser-Tyr-Ser-Met-Glu-His-Phe-Arg-Trp-Gly-Lys-
Pro-Val-NH₂
beta-MSH:
Ala-Glu-Lys-Lys-Asp-Glu-Gly-Pro-Tyr-Arg-Met-Glu-
His-Phe-Arg-Trp-Gly-Ser-Pro-Pro-Lys-Asp
gamma-MSH:
Tyr-Val-Met-Gly-His-Phe-Arg-Trp-Asp-Arg-Phe-Gly

Melanocortins exert their diverse biological effects by binding to a distinct family of receptors belonging to the G-protein coupled receptors, which display 7 transmembrane regions as a hallmark. Five melanocortin receptors have been identified (MC-1 to MC-5) corresponding to the products of 5 separate genes with a highly conserved amino acid identity. Their activation leads to elevation of intracellular cyclic adenosine monophosphate through the activation of adenylate cyclase. While ACTH activates all 5 melanocortin receptors, α-MSH activates all receptors except the MC-2 receptor. MC-1 receptor is expressed on skin keratinocytes, dendritic cells, macrophages, endothelial cells, and epithelial cells

The MC-3 and MC-4 receptors are abundantly expressed in the CNS, where they play a pivotal role in the regulation of feeding behavior and energy homeostasis. These receptors are located in several nuclei involved in the control of erectile function. Expression of the MC-3 receptor is found mainly in the hypothalamus, thalamus, brainstem, and cortex , whereas the MC-4 receptor has a wider distribution and is found essentially in all regions of the brain, including the cortex, thalamus, hypothalamus, and brainstem.

The ability of ACTH and α-MSH to cause sexual excitation has been established in different species, including rats, rabbits, cats, dogs, and monkeys. In a number of reports, cerebroventricular injection of ACTH or α-MSH in a low range (1-10 μg) has been shown to induce penile erection. The effects of melanocortins on erection appear to be androgen-dependent.

The skin is the first documented extrapituitary site of α-MSH generation and secretion, Elevated α-MSH levels are shown in several cutaneous inflammatory disorders, including psoriasis vulgaris and eczema. The potent anti-inflammatory property of α-MSH was shown in a murine model of delayed-type hypersensitivity and hapten-specific tolerance. In the latter model, α-MSH-induced hapten-specific tolerance in both a preventive as well as therapeutic treatment regimen.

In addition to skin, production and functional role of α-MSH were demonstrated in the airways in bronchoalveolar lavage fluids. Subsequently, in the animal model of allergic bronchial asthma, α-MSH suppressed allergen-specific IgE, IgG1, and IgG2a Ab production.

Furthermore, Alpha-Melanocyte-stimulating hormone ameliorates ischemic renal injury in the blood free perfused isolated rat kidney, and reduces colonic damage in a rat model of inflammatory bowel disease.

Chonic Fatigue Syndrome

Chronic fatigue syndrome (CFS) is a debilitating and complex disorder characterized by profound fatigue that is not improved by bed rest and that may be worsened by physical or mental activity. Persons with CFS most often function at a substantially lower level of activity than they were capable of before the onset of illness. In addition to these key defining characteristics, patients report various nonspecific symptoms, including weakness, muscle pain, impaired memory and/or mental concentration, insomnia, and post-exertional fatigue lasting more than 24 hours. In some cases, CFS can persist for years. The cause or causes of CFS have not been identified and no specific diagnostic tests are available. Moreover, since many illnesses have incapacitating fatigue as a symptom, care must be taken to exclude other known and often treatable conditions before a diagnosis of CFS is made. CFS occurs more often, but not exclusively, in women. CFS is most easily diagnosed when formerly active adults become ill, but it has been reported in persons of all ages, including young children and particularly teenagers.

In essence, in order to receive a diagnosis of chronic fatigue syndrome, a patient must satisfy two criteria:
1. Have severe chronic fatigue of six months or longer duration with other known medical conditions excluded by clinical diagnosis; and
2. Concurrently have four or more of the following symptoms: substantial impairment in short-term memory or concentration; sore throat; tender lymph nodes; muscle pain; multi-joint pain without swelling or redness; headaches of a new type, pattern or severity; unrefreshing sleep; and post-exertional malaise lasting more than 24 hours.

The following detailed symptoms must have persisted or recurred during six or more consecutive months of illness and must not have predated the fatigue.
Fatigue: People with CFS experience profound, overwhelming exhaustion, both mental and physical, which is worsened by exertion, and is not relieved (or not completely relieved) by rest. To receive a diagnosis of CFS, this fatigue state must last for six months.
Pain: Pain in CFS may include muscle pain, joint pain (without joint swelling or redness, and may be transitory), headaches (particularly of a new type, severity, or duration), lymph node pain, sore throats, and abdominal pain (often as a symptom of irritable bowel syndrome). Patients also report; bone, eye and testicular pain, neuralgia and painful skin sensitivity. Chest pain has been attributed variously to microvascular disease or cardiomyopathy by researchers, and many patients also report painful tachycardia.
Cognitive problems: people with CFS may experience forgetfulness, confusion, difficulty thinking, concentration difficulties, and “mental fatigue” or “brain fog”. Additional signs may be experienced; in the 2003 Canadian Definition these include aphasia, agnosia, and loss of cognitive body map.
Hypersensitivity: people with CFS are often sensitive to light, sound, and some chemicals and foods. Many CFS patients report an increase in allergic-type sensitivity to foods, scents, and chemicals, and many also report a sensitivity to medications, which can complicate treatment. Patients with pre-existing allergies, asthma, and similar conditions often report a worsening of symptoms. Sensory overload is commonly reported by patients, leading to increased fatigue and even migraine or seizures.
Poor temperature control: people with CFS often report either feeling too hot or too cold, possibly due to involvement of the hypothalamus, which regulates body temperature. Many CFS patients frequently run a low fever, or report fever-like symptoms (sweating, feeling too hot or cold, etc.) without measurable fever temperature.
Sleep problems: “Unrefreshing sleep” and rest is a hallmark of CFS, and insomnia is also common. Maintaining a sleep schedule is extremely difficult for many patients. Vivid, “feverish” dreams are a symptom in many people with CFS, exacerbating disturbed sleep patterns. Patients report that exercise, unlike in healthy persons, worsens the insomnia and unrefreshing sleep symptoms alike.
Psychological/Psychiatric symptoms: emotional lability, anxiety, depression, irritability, and sometimes a curious emotional “flattening” (most likely due to exhaustion), may manifest in CFS patients. Many of these symptoms can be directly caused by the CFS mechanism or, in some cases, may be secondary symptoms created by the syndrome, as many chronic pain or illness patients also show similar psychiatric issues. CFS patients with pre-existing psychiatric symptoms may report that these worsen with the onset of CFS. Treatment for psychiatric symptoms alone does not relieve the physical symptoms of CFS, indicating that the disease is not psychological in nature.
Disturbances in the autonomic nervous system and hormones:
   People with CFS often have abnormalities in the autonomic nervous system such as low blood volume, orthostatic intolerance, dizziness and light-headedness, especially when standing up quickly.
   Hormonal abnormalities may include abnormal vasopressin metabolism and abnormally low levels of testosterone, growth hormone and other important hormones.

Many people with CFS report a sudden, drastic start to their illness. Some people can remember a specific day or even hour when they first became ill.

One of the most common and recognizable aspects of CFS is what is called “post-exertional malaise”. When people with CFS exert themselves beyond their limits (and their limits may change daily), their symptoms worsen. The harder the exertion and the longer it lasts, the worse the symptoms will be afterward, and with greater recovery time.

People with CFS may improve after a few months, or after many years, or never at all. They may reach a plateau at some constant level of health, or may progressively decline. Often, the symptoms change over time, or cycle irregularly. Relapses are common, especially after stressful life events or additional illness. The average CFS patient is moderately to severely affected, and may expect to remain so for the duration of her or his life. It is not known whether any patients truly ‘recover’ entirely from the illness, or merely recuperate enough to regain previous levels of activity.

Treatment of CFS
Since there is no known cure for CFS, treatment is aimed at symptom relief and improved function. A combination of drug and nondrug therapies is usually recommended.
No single therapy exists that helps all CFS patients.
Lifestyle changes, including prevention of overexertion, reduced stress, dietary restrictions, gentle stretching and nutritional supplementation, are frequently recommended in addition to drug therapies used to treat sleep, pain and other specific symptoms.
Carefully supervised physical therapy may also be part of treatment for CFS. However, symptoms can be exacerbated by overly ambitious physical activity. A very moderate approach to exercise and activity management is recommended to avoid overactivity and to prevent deconditioning.
Although health care professionals may hesitate to give patients a diagnosis of CFS for various reasons, it's important to receive an appropriate and accurate diagnosis to guide treatment and further evaluation.
Delays in diagnosis and treatment are thought to be associated with poorer long-term outcomes. For example, CDC's research has shown that those who have CFS for two years or less were more likely to improve. It's not known if early intervention is responsible for this more favorable outcome; however, the longer a person is ill before diagnosis, the more complicated the course of the illness appears to be.

### DETAILED DESCRIPTION OF THE INVENTION

The invention relates to the use of the peptide α-MSH that is a free acid or pharmaceutically acceptable salt thereof that includes the sequence Ac-Ser-Tyr-Ser-Met-Glu-His-Phe-Arg-Trp-Gly-Lys-Pro-Val-NH2 for the production of a medicament for the treatment of chronic fatigue syndrome.

The invention also includes pharmaceutical compositions of matter, including α-MSH and a pharmaceutically acceptable carrier. The pharmaceutically acceptable carrier may be a buffered aqueous carrier, and preferably a saline or citrate buffered carrier.

α-MSH or pharmaceutical composition may be administered by any means known in the art, including administration by injection, administration through mucous membranes, buccal administration, oral administration, dermal administration, inhalation administration and nasal administration. In a preferred embodiment, administration is by nasal administration of a metered amount of a formulation including an aqueous buffer, which buffer may be a saline or citrate buffer, delivered by an ultrasonic nebulizer.

A primary object of the present invention is the use of α-MSH for the production of a medicament for treatment of chronic fatigue syndrome.

A primary advantage of the present invention is that it is efficacious at doses that do not cause deleterious side effects.

Yet another advantage of the present invention is that it provides α-MSH pharmaceutical for use in treatment of chronic fatigue syndrome which, because of increased efficacy at low doses, may be administered by delivery systems other than art conventional intravenous, subcutaneous or intramuscular injection, including but not limited to inhalation or nasal delivery systems via ultrasonic nebulizer and mucous membrane delivery systems.

### DESCRIPTION OF THE PREFERRED EMBODIMENTS

The peptide α-MSH may be synthesized by solid-phase means and purified to greater than 96% purity by HPLC, yielding a white powder that is a clear, colorless solution in water.

In general, α-MSH may be synthesized by solid-phase synthesis and purified according to methods known in the art. Any of a number of well-known procedures utilizing a variety of resins and reagents may be used to prepare α-MSH.

α-MSH may be in the form of any pharmaceutically acceptable salt. Acid addition salts of alpha MSH are prepared in a suitable solvent from the peptide and an excess of an acid, such as hydrochloric, hydrobromic, sulfuric, phosphoric, acetic, trifluoroacetic, maleic, succinic or methanesulfonic.

Many factors affect the stability of a pharmaceutical product, including the chemical reactivity of the active ingredient(s), the potential interaction between active and inactive ingredients, the manufacturing process, the dosage form, the container closure system, and the environmental conditions encountered during shipment, storage, handling and length of time between manufacture and usage. Pharmaceutical product stability is determined by the chemical stability as well as the physical stability of the formulation. Physical factors including heat and light may initiate or accelerate chemical reactions.

Optimal physical stability of a formulation is very important for at least three primary reasons. First, a pharmaceutical product must appear fresh, elegant and professional when it is administered to a patient. Any change in physical appearance such as color changes of haziness can cause a patient or consumer to have less confidence in the product. Second, because some products are dispensed in multiple dose containers, uniformity of dose content of the active ingredient over time must be assured. A cloudy solution or a broken emulsion can lead to a non-uniform dosage pattern. Third, the active ingredient must be available to the patient throughout the expected shelf life of the preparation. A breakdown of the product to inactive or otherwise undesired forms can lead to non-availability of the medicament to the patient.

Stability of a pharmaceutical product, then, may be defined as the capability of a particular formulation to remain within its physical, chemical, microbiological, therapeutic and toxicological specifications.

A stable solution retains its original clarity, color, and odor throughout its shelf life. Retention of clarity of a solution is a main concern in maintaining physical stability.

Solutions should remain clear over a relatively wide temperature range, such as 4° C. to about 37° C. At the lower range an ingredient may precipitate due to a lower solubility at that temperature, while at higher temperatures homogeneity may be destroyed by extractables from the glass containers or rubber closures. Thus, solutions of active pharmaceutical ingredients must be able to handle cycling temperature conditions. Similarly, a formulation should retain its color throughout this temperature range, and its odor should be stably maintained.

Small peptides are typically unstable and are susceptible to degradation in aqueous solution. In this regard, once alpha MSH has less than 90% of its labeled potency, it is no longer considered to be suitable for administration to a patient.

Various types of sugars, surfactants, amino acids and fatty acids, used singly or in combination, have been used in efforts to stabilize protein and peptide products against degradation. Wang and Hanson, J. Parenteral Science and Technology Supplement, 1988, Technical Report No. 10 describe parenteral formulations of proteins and peptides. Examples of excipients such as buffers, preservatives, isotonic agents, and surfactants are described by Manning et al., 6 Pharmaceutical Research, 1989, by Wang and Kowak, 34 J. Parenteral Drug Association 452, 1980, and Avis et al., Pharmaceutical Dosage Forms: Parenteral Medications, Vol.1, 1992.

It is understood that the development of a suitable pharmaceutical formulation for administration to a subject is complex. A need exists in the art for pharmaceutical formulations of alpha MSH designed to provide a single or multiple doses having substantial stability when refrigerated and at room temperature. Further, a need exists in the art for a liquid pharmaceutical formulation packaged with an appropriate container/closure system that also minimizes the physical and chemical degradation of such peptides.

The term buffer, buffer system, buffer solution and buffered solution, when used with reference to hydrogen-ion concentration or pH, refer to the ability of a system, particularly an aqueous solution, to resist a change of pH on adding acid or alkali, or on dilution with a solvent. Characteristics of buffered solutions, which undergo small changes of pH on addition of acid and base, in the presence either of a weak acid and a salt of the weak acid, or a weak base and a salt of the weak base. An example of the former system is citric acid and sodium citrate. The change of pH is slight as the amount of hydronium or hydroxyl ion added does not exceed the capacity of the buffer system to neutralize it.

The buffer systems can be selected from the group consisting of formate (pKa=3.75), lactate (pKa=3.86), benzoic acid (pKa=4.2) oxalate (pKa=4.29), fumarate (pKa=4.38), aniline (pKa=4.63), acetate buffer (pKa=4.76), citrate buffer (pKa2=4.76,pKa3=6.4), glutamate buffer (pKa=4.3), phosphate buffer (pKa=7.20), succinate (pKa1=4.93;pKa2=5.62), pyridine (pKa=5.23), phthalate (pKa=5.41); histidine (pKa=6.04), MES(2-(N-morpholino)ethanesulphonic acid; pKa=6.15); maleic acid (pKa=6.26); cacodylate (dimethylarsinate, pKa=6.27), carbonic acid (pKa=6.35),ADA (N-(2-acetamido)imino-diacetic acid; pKa=6.62; PIPES (4-piperazinebis-(ethanesulfonic acid; BIS-TRIS-propane (1,3-bis[tris(hydroxymethyl)methylamino]propane, pKa=6.80) pKa=6.80), ethylendiamine (pKa=6.85), ACES 2-[(2-amino-2-oxoethyl)amino]ethanesulphonic acid; pKa=6.9), imidazole (pKa=6.95), MOPS (3-(N-morphin)-propansulfonic acid; pKa=7.20), diethylmalonic acid (pKa=7.2), TES (2-[tris (hydroxymethyl) methyl] amino ethanesulphonic acid; pKa=7.50) and HEPES (N-2-hydroxylethylpiperazin-N′-2-ethansulfonic acid; pKa=7.55) buffers or other buffers having a pKa between 3.8 to 7.7 and capable of maintaining the pH of the formulation between 4.8 to 6.7.

Most preferred are buffers suitable for pharmaceutical use e.g. buffers suitable for administration to a patient such as acetate, carbonate, citrate, fumarate, glutamate, lactate, phosphate, phthalate, and succinate buffers. Particularly preferred examples of commonly used pharmaceutical buffers are acetate buffer, citrate buffer, glutamate buffer and phosphate buffer. Also in the present invention sodium chloride may be used to maintain the desired pH and thus act as the buffer component.

A stabilizer may be included in the present formulation but, and importantly, is not needed. If included, however, a stabilizer useful in the practice of the present invention is a carbohydrate or a polyhydric alcohol or a chelating agent. A suitable carbohydrate or polyhydric alcohol useful in practice of the present invention is about 1 to 10% (w/v) of a pharmaceutical composition. A suitable chelating agent is approximately 0.04 to 0.2% of the pharmaceutical formulation.

The polyhydric alcohols and carbohydrates share the same chemical feature, i.e., —CHOH—CHOH—, which is responsible for stabilizing peptides and proteins. The polyhydric alcohols include such compounds as sorbitol, mannitol, glycerol, inositol, xylitol, and polypropylene/ethylene glycol copolymer, as well a various polyethylene glycols (PEGs) of molecular weight 200, 400, 1450, 3350, 4000, 6000, and 8000. These molecules are straight chain molecules. The carbohydrate, such as mannose, ribose, trehalose, maltose inositol, erythritol and lactose are cyclic molecules which may contain a keto or aldehyde group. These two classes of compounds have been demonstrated to be effective in stabilizing peptides and proteins against denaturation caused by elevated temperatures and by freeze-thaw or freeze-drying processes and against degradation.

A chelating agent used in practice is EDTA (ethylene-diaminetetraacetate) and derivatives. It is a stabilizer used in drugs and cosmetics to prevent ingredients in a given formula from binding with trace elements (particularly minerals) that can exist in water and other ingredients to cause unwanted product changes such as texture, odor, and consistency problems. In particular, it has been shown that trace amounts of heavy metals accelerate the natural hydrolysis of peptides and proteins. Sorbitol and mannitol are the preferred polyhydric alcohols. Another useful feature of the polyhydric alcohols is the maintenance of the tonicity of the lyophilized formulations described herein.

The United States Pharmacopoeia (USP) states that antimicrobial agents in bacteriostatic and fungistatic concentration must be added to preparations contained in multiple dose containers. They must be present in adequate concentration at the time of use to prevent the multiplication of microorganisms inadvertently introduced into the preparation while withdrawing a portion of the content with a hypodermic needle and syringe, or using other invasive means for delivery, such a pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular agent will be effective in one formulation but not effective in another formulation.

A preservative is, in the common pharmaceutical sense, a substance that prevents or inhibits microbial growth and may be added to pharmaceutical formulation for this purpose to avoid consequent spoilage of the formulation by microorganisms. While the amount of the preservative is not large, it may nevertheless affect the overall stability of the peptide, thus even selection of preservative can be difficult.

While the preservative for use in the practice of the present invention can range from 0.005 to 1% (w/v), the preferred range for each preservative, alone or in combination with other is benzyl alcohol (0.2-1%), or m-cresol (0.1-0.3%, or phenol (0.1-0.8%) or combination of methyl (0.05-0.25%) and ethyl or propyl or butyl (0.005%-00.3%) parabens. The parabens are lower alkyl esters of parahydroxybenzoic acid.

α-MSH has a tendency to adsorb onto the glass in a glass container when in liquid formulation, therefore, a surfactant can further stabilize the pharmaceutical formulation. Surfactants frequently cause denaturation of protein, both by hydrophilic disruption and by salt bridge separation. Relatively low concentrations of surfactants exert potent denaturing activity, because of the strong interactions between surfactant moieties and the reactive sites on proteins. However, judicious use of this interaction can stabilize peptides and proteins against interfacial or surface denaturation and absorption. Surfactant which could further stabilize the peptide may optionally be present in the range of about 0.001 to 0.3% (w/v) of the total formulation and include poly sorbate 80 (i.e., polyoxyethylene(20) sorbitan monooleate; Tween 80), CHAPS® (i.e., 3-[(3-cholamidopropyl) dimethylammonio]1-propansulfonate), Brij® (e.g., Brij 35, which is (polyoxyethylene (23) lauryl ether), poloxamer, or another non-ionic surfactant.

It is also possible that other ingredients may be present in the peptide pharmaceutical formulation of the present invention. Such additional ingredients may include wetting agents, emulsifiers, bulking agents, tonicity modifier, metal ions, oleaginous vehicles, proteins (e.g. human serum albumin, gelatin) and zwitterions (e.g. an amino acid such as betaine, taurine, arginine, glycine, lysine and histidine). Such additional ingredients, of course should not adversely affect the overall stability of the pharmaceutical formulation of the present invention.

The vehicle of greatest importance for parenteral drugs and drugs for inhalation is water. The water of suitable quality for inhaled administration must be prepared either by distillation or by reverse osmosis. Only by these means is it possible to separate adequately various liquid, gas and solid contaminating substances from water. Water for injection is the preferred aqueous vehicle for use in the pharmaceutical formulation of the present invention.

Containers are also an integral part of the formulation of an inhalation or injection and may be considered a component, for there is no container that is totally insoluble or does not in some way affect the liquid it contains, particularly if the liquid is aqueous. Therefore, the selection of a container for an inhaled or parenteral pharmaceutical formulation must be based on a consideration of the composition of the container, as well as of the solution, and the treatment to which it will be subjected. Adsorption of the peptide to the glass surface of the vial can also be minimized by use of borosilicate glass, for example FIOLAX®o.c.-Klar glass (Schott, Germany), Wheaton-33® low extractable borosilicate glass (Wheaton Glas Co.,USA) or Corning® Pyrex® 7740 (Corning Inc., USA). Other glass types which can be used e.g. colorless glass, hydrolytic class I plus; 6 R according to DIN ISO 8362 (Schott, St. Gallen, Switzerland), are supposed to meet the criteria of type I borosilicate glass of ASTM (American Society for Testing and Materials), EP (European Pharmacopoeia), and USP (United States Pharmacopoeia). For example, the biological and chemical properties of Alpha MSH is stabilized by formulation and lyophilization in a FIOLAX®o.c.-Klar borosilicate glass vial to a final concentration of 0.033 mg/ml and 2 mg/ml of Alpha MSH in the presence of 5% mannitol and 0.02% Tween 80.

Stoppers for glass vials, such as, Teflon coated rubber stopper 20 mm, FM259/0 dark grey (Ph.Eur. type I) (Helvoet Pharma, Alken, Belgium) or red injection rubber stoppers 20 mm V9034, (Helvoet Pharma, Alken, Belgium) or any equivalent stopper can be used as the closure for pharmaceutical formulation for inhalation or injection.

Any sterilization process can be used in developing the peptide pharmaceutical formulation of the present invention. Typical sterilization processes include filtration, steam (moist heat), dry heat, gases (e.g., ethylene oxide, formaldehyde, chlorine dioxide, propylene oxide, betapropiolactone, ozone, chloropierin, peracetic acid methyl bromide and the like), radiant exposure and aseptic handling. Filtration is the preferred method of sterilization in the practice of the present invention. The sterile filtration involves filtration through 0.22 μm filter. After filtration, the solution is filled into appropriate vials as described above.

The α-MSH formulation of the present invention may also be lyophilized (freeze-dried). The lyophilized product can then be rehydrated before use.

The formulation of the present invention is preferably intended for inhaled or intranasal administration. Other possible routes of administration include intramuscular, intravenous, intracavernous, subcutaneous, intradermal, intraarticular, intrathecal, mucosal and the like.

The current invention describes the process and methods for manufacturing of a soluble pharmaceutical composition containing α-MSH comprising the following steps:
1.Generation of a buffer system which is capable of maintaining the pH value between 4.6 and 6.9 in the absence of a pharmaceutically active amount of α-MSH and at least one stabilizer
2.Addition of a pharmaceutically active amount of alpha MSH and at least one stabilizer to such buffer.

Preferably, the buffer is an aqueous, or mostly aqueous buffer. The term “mostly aqueous” means that organic solvents can be added up to 15% per volume, preferably up to 10% per volume of the aqueous buffer. Suitable organic solvents are ethanol and/or isopropanol. Further it was found to be advantageous to add at least one stabilizer to the solution containing Alpha MSH. Particularly useful stabilizers comprise EDTA and/or mannitol or sorbitol.

Routes of Administration

α-MSH may be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. In general, any route of administration by which a-MSH is introduced across an epidermal layer of cells may be employed. Administration means may include administration through mucous membranes, buccal administration, oral administration, dermal administration, inhalation administration, nasal administration and the like. The dosage for treatment of chronic fatigue syndrome is administration, by any of the foregoing means or any other means known in the art, of an amount sufficient to improve quality of life of the sufferer.

In general, the actual quantity of α-MSH administered to a patient will vary between fairly wide ranges depending upon the mode of administration, and the formulation used.

Nasal or Intrapulmonary Administration.

By “nasal administration” is meant any form of intranasal administration of α-MSH. α-MSH may be in an aqueous solution, such as a solution including saline, citrate or other common excipients or preservatives. Preferably, respective aerosols are produced by ultrasonic nebulizer and delivered at a-MSH may also be in a dry or powder formulation.

In an alternative embodiment, a-MSH may be administered directly into the lung. Intrapulmonary administration may be performed by means of a metered dose inhaler, a device allowing self-administration of a metered bolus of a peptide of this invention when actuated by a patient during inspiration.

α-MSH may be formulated with any of a variety of agents that increase effective nasal absorption of drugs. These agents should increase nasal absorption without unacceptable damage to the mucosal membrane. U.S. Pat. Nos. 5,693,608, 5,977,070 and 5,908,825, among others, teach a number of pharmaceutical compositions that may be employed, including absorption enhancers, and the teachings of each of the foregoing, and all references and patents cited therein, are incorporated by reference.

If in an aqueous solution, α-MSH may be appropriately buffered by means of saline, acetate, phosphate, citrate, acetate or other buffering agents, which may be at any physiologically acceptable pH, generally from about pH 4 to about pH 7. A combination of buffering agents may also be employed, such as phosphate buffered saline, a saline and acetate buffer, and the like. In the case of saline, a 0.9% saline solution may be employed. In the case of acetate, phosphate, citrate, acetate and the like, a 50 mM solution may be employed. In addition to buffering agents, a suitable preservative may be employed, to prevent or limit bacteria and other microbial growth. One such preservative that may be employed is 0.05% benzalkonium chloride.

It is also possible and contemplated that α-MSH may be in a dried and particulate form. In a preferred embodiment, the particles are between about 0.5 and 6.0 micrometers, such that the particles have sufficient mass to settle on the lung surface, and not be exhaled, but are small enough that they are not deposited on surfaces of the air passages prior to reaching the lung. Any of a variety of different techniques may be used to make dry powder microparticles, including but not limited to micro-milling, spray drying and a quick freeze aerosol followed by lyophilization. With micro-particles, the peptides may be deposited to the deep lung, thereby providing quick and efficient absorption into the bloodstream. Further, with such approach penetration enhancers are not required, as is sometimes the case in transdermal, nasal or oral mucosal delivery routes. Any of a variety of inhalers can be employed, including propellant-based aerosols, nebulizers, single dose dry powder inhalers and multidose dry powder inhalers. Common devices in current use include metered dose inhalers, which are used to deliver medications for the treatment of asthma, chronic obstructive pulmonary disease and the like. Preferred devices include dry powder inhalers, designed to form a cloud or aerosol of fine powder with a particle size that is always less than about 6.0 .mu.m. One type of dry powder inhaler in current use is Glaxo's Rotahaler.TM., which dispenses a unit dose of powder into a tube, and employs patient suction for inhalation of the powder. Other, more advanced and preferred dry powder inhalers have been or are in development, which include propellants and the like.

Microparticle size, including mean size distribution, may be controlled by means of the method of making. For micro-milling, the size of the milling head, speed of the rotor, time of processing and the like control the microparticle size. For spray drying, the nozzle size, flow rate, dryer heat and the like control the microparticle size. For making by means of quick freeze aerosol followed by lyophilization, the nozzle size, flow rate, concentration of aerosoled solution and the like control the microparticle size. These parameters and others may be employed to control the microparticle size.

In one preferred embodiment, a dry powder inhaler is employed which includes a piezoelectric crystal that deaggregates a dry powder dose, creating a small powder “cloud.” Once the powder cloud is generated, an electricostatically charged plated above the powder cloud lifts the drug into the air stream. The user with one relatively easy breath can then inhale the powder. The device may be breath activated, utilizing a flow sensor that activates the electronic components upon the start of inhalation, and thereby eliminating the need for coordination of activation and breathing rhythms by the user.

The pharmaceutical compositions according to the current invention are suitable for the manufacturing of a medicament for the prophylaxis and/or treatment of chronic fatigue syndrome.

The medicaments of the invention are preferentially formulated for inhalative or intranasal administration. Suitable protocols for the administration of the inventive alpha MSH formulations are presented in Examples.

Furthermore, the preferred soluble pharmaceutical compositions are prepared in a sterile form.

### EXAMPLE

The following provides clinical example for drug dosages, safety and efficacy for inhaled administration by chronically ill patient of the medicament formulation.

A female, 34 year old patient suffering from chronic fatigue syndrome showed extremely low levels of α-MSH in her blood serum (<8 pg/ml—normally 30-40 pg/ml). α-MSH concentrations in serum was determined using a competitive RIA. α-MSH in samples competed with ¹²⁵I-labeled α-MSH in binding to an antiserum, which was raised against an α-MSH-albumin conjugate. To increase the sensitivity of the assay, ¹²⁵I-α-MSH was added delayed. Ab-bound ¹²⁵I-α-MSH was separated from the free fraction using the double Ab polyethylene glycol precipitation technique. The radioactivity of the precipitate was measured. The antiserum used in this assay was directed to the C-terminal part of the α-MSH molecule and showed no cross-reactivity with adrenocorticotropic hormone. Briefly, 100 μl of samples was pipetted in 3-ml glass tubes, 200 μl anti-α-MSH serum was added, and the mixture was incubated for 24 h at 4° C. On the following day, 200 μl ¹²⁵I-α-MSH was added and the mix was incubated for further 24 h at 4° C. On day 3, double Ab polyethylene glycol (500 μl) was added and the mixture was incubated for another 60 min. Finally, vials were centrifuged, supernatants were decanted, and the radioactivity was measured in the precipitates using a gamma counter (counting time, 3 min).

The patient experienced terribly pulmonary stress when exercising on a bicycle, including increase of pulmonary arterial pressure. Spirometry testing revealed that her FEV1 was 2,2 I.

After inhaling 600 micrograms α-MSH per day, split into 3 equal doses for 4 consecutive weeks using an ultrasonic nebulizer, the patient significantly improved as measured by the SF-36 quality of life questionnaire and by improving the FEV1 value to 2,6 I. The physical and clinical improvement was not accompanied by any negative side effects.

1. Use of the peptide a-MSH for manufacturing of a medicament for treatment of chronic fatigue syndrome.

2. Use of the peptide a-MSH for manufacturing of a medicament for treatment of chronic fatigue syndrome and associated neurodegenerative disorders,

3. Use according to claim 1, where the medicament is administered by an ultrasonic nebulizer.

4. Use according to claim 2, where the medicament is administered by an ultrasonic nebulizer.
